(19) United States
(12) Patent Application Publication
(10) Pub. No.: US 2026/0216273 A1
(43) Pub. Date: Jul. 30, 2026

(54) ANIMAL TREATMENT FORMULATION AND METHOD

(71) Applicant: AUSSAN LABORATORIES PTY LTD, CAMPBELLFIELD, Victoria (AU)

(72) Inventors: Kevin John JOYCE, Chatswood (AU); Robert WATSON, Campbellfield (AU)

(73) Assignee: AUSSAN LABORATORIES PTY LTD, CAMPBELLFIELD, Victoria (AU)

(21) Appl. No.: 19/152,287

(22) PCT Filed: Jan. 31, 2024
(86) PCT No.: PCT/AU2024/050058

§ 371 Date: Jul. 30, 2025

(30) Foreign Application Priority Data
Jan. 31, 2023 (AU) 2023900222

(51) Int. Cl. A61K 36/752; A61K 9/00; A61K 9/12; A61K 31/191; A61K 31/352; A61K 31/375; A61P 31/04; A61P 31/20

(52) U.S. Cl. CPC A61K 36/752; A61K 9/0014; A61K 9/12; A61K 31/191; A61K 31/352; A61K 31/375; A61P 31/04; A61P 31/20

(57) ABSTRACT

Accordingly, in one aspect of the present invention there is provided a method of treating an animal having a viral and microbial skin infection comprising: creating a formulation comprising a mixture of between 10-40% by weight of bioflavonoids; 2-10% by weight of ascorbic acid; 5-20% by weight of lactic acid; and 5-20% by weight of an organic surfactant; diluting the formulation to form a treatment solution; and applying the treatment solution to a surface of the animal having the infection.

### RELATED APPLICATION(S)

The present application claims priority from Australian Provisional Patent Application No. 2023900222, the entire contents of which are incorporated herein by reference.

### FIELD OF INVENTION

The present invention relates generally to a formulation and method for the treatment of diseases in animals, and in particular, to a method and formulation for the treatment of viral, fungal and bacterial diseases in animals, such as bovines.

### BACKGROUND OF THE INVENTION

Diseases in animals often require invasive or costly interventions that can lead to ongoing detrimental effects, especially in agriculture livestock where an unhealthy animal impacts the income of the farmer and can cause other animals to contract the disease.

Whilst many such diseases may have a relatively low mortality, they have a significant impact on the health of the animals and can significantly reduce milk yield and animal reproduction. With food production becoming an increasingly important resource to manage, it is fundamental that management of skin diseases and wounds be improved to ensure that the food needs of the population are being met and that this is done in an efficient and safe manner for the animal, the farmer and the consumer.

Skin diseases, such as Lumpy skin disease (LSD), are highly contagious viral diseases typically found in bovines, such as cattle and buffalo. Typically, in situations where an animal becomes infected with LSD, the animal will develop a fever and characteristic skin nodules that create large holes in the animal's skin that are prone to infection. The disease can be transferred between infected animals through insects, such as mosquitoes and flies, and also through contact between infected animals and contaminated items, such as animal handling equipment, livestock vehicles, as well as people's clothing and footwear.

For diseases such as LSD, there is no specific treatment for animals infected with the disease. Infected animals are typically given antibiotics and treatment for their lesions and fever, and isolated from other animals to limit transmission of the disease.

As such, there is a need to provide a means for treating infected animals that can be applied to infected animals to inactivate the virus which is convenient and not harmful to the animal and the environment.

The above references to and descriptions of prior proposals or products are not intended to be, and are not to be construed as, statements or admissions of common general knowledge in the art. In particular, the above prior art discussion does not relate to what is commonly or well known by the person skilled in the art, but assists in the understanding of the inventive step of the present invention of which the identification of pertinent prior art proposals is but one part.

### STATEMENT OF INVENTION

The invention according to one or more aspects is as defined in the independent claims. Some optional and/or preferred features of the invention are defined in the dependent claims.

Accordingly, in one aspect of the present invention there is provided a method of treating an animal having a viral and microbial skin infection comprising:
creating a formulation comprising a mixture of between 10-40% by weight of bioflavonoids; 2-10% by weight of ascorbic acid; 5-20% by weight of lactic acid; and 5-20% by weight of an organic surfactant;
diluting the formulation to form a treatment solution; and
applying the treatment solution to a surface of the animal having the infection.

The step of creating the formulation may comprise sourcing the bioflavonoids from the group consisting of citrus fruit plant extracts, including grapefruit, lemon, lime, orange, tangerine, pomelo, bitter orange extract.

The step of diluting the formulation may comprise mixing the formulation with water to dilute the formulation between 0.5-10%.

The formulation may be diluted with water to achieve a treatment solution with a dilution of around 1%.

Ion one embodiment, the treatment solution is applied to the skin of the animal in the form of a spray or mist. In another embodiment, the treatment solution may be applied to the skin of the animal in the form of a dip into which the animal is fully or partially submerged.

In one application, the treatment may be applied to the teats of the animal as a method of treating mastitis in the animal.

In another application the treatment solution may be applied to open wounds on the animals skin to treat Lumpy Skin Disease (LSD).

Accordingly, in another aspect of the present invention there is provided a formulation for application to an animal for treating a viral skin condition comprising: a mixture of between 10-40% by weight of bioflavonoids; 2-10% by weight of ascorbic acid; 5-20% by weight of lactic acid; and 5-20% by weight of an organic surfactant.

### DETAILED DESCRIPTION

The present invention will be described below in relation to a formulation and method of applying a formulation to an animal infected with a viral skin disease, such as Lumpy Skin Disease (LSD) and a bacterial disease such as mastitis. However, it will be appreciated that the formulation and method of application of the present invention could be applied to treat a variety of different viral and microbial diseases in a variety of different animals and still fall within the scope of the present invention.

The present invention has been tested in laboratories as a topical treatment on cows suffering from illnesses such as Lumpy Skin Disease and mastitis. It has also been applied to open wounds in their skin.

The formulation of the present invention comprises an anti-microbial agent being a synergistic mixture of bioflavonoids together with certain organic acids and carriers. Such a formulation may also include alcohol, surfactant, glycerine and water.

The formulation will be provided by using a mixture of the invention diluted with water between 0.5-10% dilution, preferably around 1%.

The bioflavonoid portion of the formulation may be selected from the group consisting of citrus fruit plant extracts, including grapefruit, lemon, lime, orange, tangerine, pomelo, bitter orange extract.

The organic surfactant portion of the formulation may be selected from any known organic surfactants typically made from fatty acid esters of sugars and fatty acid esters or amides of amino acids, including acylpolyols, glycolipids and the like,

In accordance with a first embodiment of the present invention, the formulation may be configured to comprise the ingredients by percentage, as follows:

| Ingredient | % by weight |
|---|---|
| Bioflavonoids | 10-40% |
| ascorbic acid | 2-10% |
| Lactic acid | 5-20% |
| Organic Surfactant | 5-20% |

As mentioned above, the formulation for use is diluted and the amount of dilution may be dependent on the viral or microbial load carried by the animal to be treated. In some instances, where an animal carries a high load, dilution can be very high (40 ml/litre). For other applications, we have found that between 0.5 and 1.5 ml per litre of the mixture added to water provides a satisfactory dilution.

The formulation may be applied directly to the skin of the animal in the form of a spray, such as a light mist. It may also be used in the form of a dip into which the animals are fully or partially submerged. In other embodiments, the formulation can be administered orally or intravenously. As bioflavonoids are commonly used as human dietary supplements, they become mobile in the blood stream and are able to latch on to the virus or microbe to render it inert and prevent replication.

Due to the configuration of the formulation, as the residues of the product components are not toxic and are applied in only small quantities, all ingredients are natural products which are acceptable for use in food and organic farming applications without undue side effects on the animals or the environment. In any case, because of the low concentration levels of the formulation, the amount of residues is extremely small.

To support the effectiveness of the formulation in inactivating the LSD virus, a test was carried out by applying the formulation on an inanimate surface containing the LSD virions.

The residual viral infectivity of the inanimate surface was quantified by determination of Tissue Culture Infective Dose 50 (TCID₅₀) in Vero cells. The cytotoxic concentration 50 (CC₅₀) of the formulation in Vero Cells was determined to be 0.05%.

The following results were obtained by such tests:

| Formulation | % Inactivation |
|---|---|
| Concentration | of LSDV |
| (% v/v) | Virions |
| 1% | 99.99% |
| 0.1% | 68.09% |
| 0.01% | 13.48% |

The results show that the formulation having a 1% concentration was effective in inactivating 99.99% of the LSD Virions present on an inanimate surface.

To support the effectiveness of the formulation in inactivating the bacteria that cause mastitis, a test was carried out by applying the formulation on specified test microorganisms within a given contact time in a suspension, these being Escherichia coli and Staphylococcus.

Testing was conducted under the BS EN 1276:2009; chemical disinfectants and antiseptics-quantitative suspension test for the evaluation of bactericidal activity.

The following results were obtained by such tests:

| Formulation | % | % |
|---|---|---|
| Concentration | Inactivation | Inactivation of |
| (% v/v) | of E-Coli | Staphylococcus |
| 0.5% | >99.999 | 99.99% |

The results show that the formulation having a 0.5% concentration was effective in inactivating over 99.99% of the bacteria present, thereby effectively treating an animal, such as a bovine having mastitis.

The formulation has also proven successful in the prevention and treatment of disease in calves and significantly reducing, and in some cases eliminating, mortality issues in such animals over the first weeks of life. The formulation is typically applied to the new-born animals by spraying and fogging the animals following birth and in the first weeks of life. For such animals, their skin is coated with the formulation during this period.

Throughout the specification and claims the word “comprise” and its derivatives are intended to have an inclusive rather than exclusive meaning unless the contrary is expressly stated or the context requires otherwise. That is, the word “comprise” and its derivatives will be taken to indicate the inclusion of not only the listed components, steps or features that it directly references, but also other components, steps or features not specifically listed, unless the contrary is expressly stated or the context requires otherwise.

It will be appreciated by those skilled in the art that many modifications and variations may be made to the methods of the invention described herein without departing from the spirit and scope of the invention.

1. A method of treating an animal having a viral and microbial skin infection comprising:
creating a formulation comprising a mixture of between 10-40% by weight of bioflavonoids; 2-10% by weight of ascorbic acid; 5-20% by weight of lactic acid; and 5-20% by weight of an organic surfactant;
diluting the formulation to form a treatment solution; and
applying the treatment solution to a surface of the animal having the infection.

2. A method according to claim 1, wherein the step of creating the formulation comprises sourcing the bioflavonoids from the group consisting of citrus fruit plant extracts, including grapefruit, lemon, lime, orange, tangerine, pomelo, bitter orange extract.

3. A method according to claim 1, wherein the step of diluting the formulation comprises mixing the formulation with water to dilute the formulation between 0.5-10%.

4. A method according to claim 3, wherein the formulation is diluted with water to achieve a treatment solution with a dilution of around 1%.

5. A method according to claim 1, wherein the treatment solution is applied to the skin of the animal in the form of a spray or mist.

6. A method according to claim 1, wherein the treatment solution is applied to the skin of the animal in the form of a dip into which the animal is fully or partially submerged.

7. A method according to claim 5, wherein the treatment is applied to the teats of the animal to treat mastitis.

8. A method according to claim 1, wherein the treatment solution is applied to open wounds on the animals skin to treat Lumpy Skin Disease (LSD).

9. A formulation for application to an animal for treating a viral and microbial skin infections comprising: a mixture of between 10-40% by weight of bioflavonoids; 2-10% by weight of ascorbic acid; 5-20% by weight of lactic acid; and 5-20% by weight of an organic surfactant.
